(19) United States
(12) Patent Application Publication
(10) Pub. No.: US 2024/0058003 A1
(43) Pub. Date: Feb. 22, 2024

(54) ANVIL BUTTRESS LOADING SYSTEM FOR SURGICAL STAPLING APPARATUS

(71) Applicant: Covidien LP, Mansfield, MA (US)

(72) Inventors: Kenneth H. Whitfield, North Haven, CT (US); Roanit A. Fernandes, Hamden, CT (US)

(21) Appl. No.: 18/386,657

(22) Filed: Nov. 3, 2023

Related U.S. Application Data
(63) Continuation of application No. 17/533,745, filed on Nov. 23, 2021, now Pat. No. 11,806,017.

(51) Int. Cl. A61B 17/072

(52) U.S. Cl. CPC A61B 17/07292; A61B 2017/0053

(57) ABSTRACT

A surgical buttress loading system includes an anvil having a spring assembly biased against a tissue facing surface of an anvil body and a loading tool including a cavity configured to receive the anvil therein, a buttress cartridge including a surgical buttress releasably disposed thereon, an actuator, and a handle operably coupled to the buttress cartridge. When the anvil is positioned within the cavity of the loading tool and the handle is moved to from an unactuated position to an actuated position, the handle moves the actuator to from a retracted position to an extended position and into engagement with the spring assembly of the anvil to lift the spring assembly off of the tissue facing surface and then slides the buttress cartridge towards the spring assembly to move a proximal end portion of the surgical buttress between the spring assembly and the tissue facing surface.

### FIELD

This application generally relates to surgical stapling apparatus, and more particularly, to surgical buttress loading tools for releasably securing surgical buttresses to anvil assemblies of the surgical stapling apparatus.

### BACKGROUND

Surgical stapling apparatus are employed by surgeons to sequentially or simultaneously apply one or more rows of fasteners, e.g., staples or two-part fasteners, to body tissue for the purpose of joining segments of body tissue together. Such apparatus generally include a pair of jaws or finger-like structures between which the body tissue to be joined is placed. When the surgical stapling apparatus is actuated, or “fired”, longitudinally moving firing bars contact staple drive members in one of the jaws. The staple drive members push the surgical staples through the body tissue and into an anvil in the opposite jaw which forms the staples. If body tissue is to be removed or separated, a knife blade can be provided in the jaws of the apparatus to cut the body tissue between the lines of staples.

Surgical supports, e.g., meshes or buttress materials, may be used in combination with surgical stapling apparatus to bridge, repair, and/or reinforce tissue defects within a patient. A clinician may manually attach the buttress materials to the surgical stapling apparatus in the operating room during a surgical procedure, or utilize a surgical stapling apparatus including buttress materials pre-installed thereon. The buttress material reinforces the staple or suture line as well as covers the juncture of the tissues to reduce leakage prior to healing.

### SUMMARY

This disclosure relates to anvil side (single-sided) buttress attachment onto a loading unit of a surgical stapling apparatus. The surgical buttress loading tools of this disclosure are designed to make surgical buttress attachment (e.g., in the operating room) a simple, straightforward, and cost-effective procedure.

In one aspect, the disclosure provides a surgical buttress loading system including an anvil and a surgical buttress loading tool. The anvil includes an anvil body including a spring assembly biased against a tissue facing surface of the anvil body. The surgical buttress loading tool includes a first housing portion, a second housing portion, a buttress cartridge, an actuator, and a handle. The first and second housing portions define a cavity therebetween that is configured to receive the anvil therein. The buttress cartridge is supported on the first housing portion and includes a surgical buttress releasably disposed thereon. The actuator is disposed within a proximal end portion of the second housing portion and is movable between retracted and extended positions. The handle is supported on the second housing portion and is operably coupled to the buttress cartridge. The handle is slidable between unactuated and actuated positions. When the anvil is positioned within the cavity of the surgical buttress loading tool and the handle is moved from the unactuated position to the actuated position, the handle moves the actuator from the retracted position to the extended position and into engagement with the spring assembly of the anvil to lift the spring assembly off of the tissue facing surface and then slides the buttress cartridge towards the spring assembly to move a proximal end portion of the surgical buttress between the spring assembly and the tissue facing surface. When the handle is moved back to the unactuated position, the actuator moves back to the retracted position and disengages from the spring assembly to releasably secure the proximal end portion of the surgical buttress to the anvil.

The spring assembly may include springs that each includes an arm extending over a through-hole defined in the anvil, and the actuator may include pins aligned with the through-holes when the anvil is positioned within the cavity of the surgical buttress loading tool. When the actuator is moved to the extended position, the pins of the actuator may extend through the though-holes and push the arms of the springs away from the tissue facing surface of the anvil plate.

In aspects, the anvil includes an anvil tip extending distally from the anvil body. The anvil tip may include a hook and when the handle is moved to the actuated position, the buttress cartridge may slide a distal end portion of the surgical buttress over the hook to releasably secure the distal end portion of the surgical buttress to the anvil. In some aspects, the distal end portion of the surgical buttress includes a window defined therein, and the hook extends through the window.

The handle may include a tab extending into an elongated slot of the buttress cartridge, and when the handle is moved to the actuated position, the tab may initially move within the elongated slot while the handle moves the actuator to the extended position before sliding the buttress cartridge. The handle may include a sloped proximal end positioned adjacent to a ramped surface of the actuator and when the handle is moved to the actuated position, the sloped proximal end may cam against the ramped surface.

In aspects, the surgical buttress loading tool includes an applicator spring secured to the second housing portion. The applicator spring includes a spring leg biasing the actuator in the retracted position. In some aspects, the applicator spring includes a spring arm holding the surgical buttress against the buttress cartridge until the anvil is positioned into the cavity of the surgical buttress loading tool.

The surgical buttress loading tool may include an interlock disposed within the second housing portion. The interlock may be configured to lock the handle from movement until the anvil is positioned within the cavity of the surgical buttress loading tool. In aspects, the interlock is laterally movable between a locked position and an unlocked position and when the interlock is in the locked position, a first arm of the interlock is engaged with a notch defined in the handle to prevent movement of the handle between the unactuated and actuated positions. In some aspects, the interlock includes a second arm extending partially into the cavity defined between the first and second housing portions and, when the anvil is inserted into the cavity, the anvil moves the interlock from the locked position to the unlocked position. In certain aspects, the interlock includes a leg and when the interlock is in the locked position, the leg is engaged with a notch defined in the buttress cartridge to prevent movement of the buttress cartridge.

In another aspect, the disclosure provides a method of loading a surgical buttress onto an anvil including: inserting an anvil into a cavity of a surgical buttress loading tool, the anvil including an anvil body including a spring assembly biased against a tissue facing surface of the anvil body and the anvil buttress loading tool including first and second housing portions defining the cavity therebetween, a buttress cartridge supported on the first housing portion and including a surgical buttress releasably disposed thereon, an actuator disposed within a proximal end portion of the second housing portion, the actuator movable between retracted and extended positions, and a handle supported on the second housing portion and operably coupled to the buttress cartridge, the handle slidable between unactuated and actuated positions; moving the handle from the unactuated position to the actuated position so that the handle moves the actuator from the retracted position to the extended position and into engagement with the spring assembly of the anvil to lift the spring assembly off of the tissue facing surface and then slides the buttress cartridge towards the spring assembly to move a proximal end portion of the surgical buttress between the spring assembly and the tissue facing surface; and moving the handle back to the unactuated position so that the actuator moves back to the retracted position and disengages from the spring assembly to releasably secure the proximal end portion of the surgical buttress to the anvil.

According to yet another aspect of the disclosure, a surgical buttress loading tool for use with an anvil including an anvil body having a spring assembly biased against a tissue facing surface of the anvil body, is provided. The surgical buttress loading tool includes first and second housing portions defining a cavity therebetween, the cavity configured to receive the anvil therein; a buttress cartridge supported on the first housing portion and including a surgical buttress releasably disposed thereon; an actuator disposed within a proximal end portion of the second housing portion, the actuator movable between retracted and extended positions; and a handle supported on the second housing portion and operably coupled to the buttress cartridge, the handle slidable between unactuated and actuated positions.

In use, when the anvil is positioned within the cavity of the surgical buttress loading tool and the handle is moved from the unactuated position to the actuated position, the handle moves the actuator from the retracted position to the extended position and into engagement with the spring assembly of the anvil to lift the spring assembly off of the tissue facing surface and then slides the buttress cartridge towards the spring assembly to move a proximal end portion of the surgical buttress between the spring assembly and the tissue facing surface. Further, when the handle is moved back to the unactuated position, the actuator moves back to the retracted position and disengages from the spring assembly to releasably secure the proximal end portion of the surgical buttress to the anvil.

The handle may include a tab extending into an elongated slot of the buttress cartridge. In use, when the handle is moved to the actuated position, the tab may initially move within the elongated slot while the handle moves the actuator to the extended position before sliding the buttress cartridge.

The handle may include a sloped proximal end positioned adjacent to a ramped surface of the actuator. In use, when the handle is moved to the actuated position, the sloped proximal end may cam against the ramped surface.

The surgical buttress loading tool may further include an applicator spring secured to the second housing portion. The applicator spring may include a spring leg biasing the actuator in the retracted position.

The applicator spring may include a spring arm holding the surgical buttress against the buttress cartridge until the anvil is positioned into the cavity of the surgical buttress loading tool.

The surgical buttress loading tool may further include an interlock disposed within the second housing portion. The interlock may be configured to lock the handle from movement until the anvil is positioned within the cavity of the surgical buttress loading tool.

The interlock may be laterally movable between a locked position and an unlocked position and when the interlock is in the locked position, a first arm of the interlock is engaged with a notch defined in the handle to prevent movement of the handle between the unactuated and actuated positions.

The details of one or more aspects of the disclosure are set forth in the accompanying drawings and the description below. Other aspects, as well as features, objects, and advantages of the aspects described in this disclosure will be apparent from the description and drawings, and from the claims.

### BRIEF DESCRIPTION OF DRAWINGS

Various aspects of the disclosure are described hereinbelow with reference to the drawings, which are incorporated in and constitute a part of this specification, wherein:

FIG. 1 is a perspective view of a surgical stapling apparatus in accordance with an aspect of the disclosure;

FIG. 2 is perspective view of a tool assembly of the surgical stapling apparatus of FIG. 1;

FIG. 3 is a perspective view of an anvil of the tool assembly of FIG. 2;

FIG. 4 is a close-up view of area of detail 4 in FIG. 3, illustrating a spring assembly of the anvil;

FIG. 5 is a partial, perspective view of the anvil of FIG. 3, shown with the spring assembly separated from the anvil;

FIG. 6 is a partial, perspective view of an anvil tip of the anvil of FIG. 3;

FIG. 7 is a perspective view of a loading tool, shown in a closed position, in accordance with aspects of the disclosure;

FIG. 8 is a perspective view of the loading tool of FIG. 7, shown in an open position;

FIG. 9 is a perspective view of the loading tool of FIG. 7, shown with parts separated;

FIG. 10 is a close-up view of the area of detail 10 in FIG. 9, illustrating an actuator of the loading tool;

FIG. 11 is a close-up view of the area of detail 11 in FIG. 9, illustrating an interlock of the loading tool;

FIG. 12 is a perspective view of the loading tool of FIG. 8, shown in an open position with a buttress cartridge separated from a first housing body;

FIG. 13 is a perspective view of the loading tool of FIG. 12, shown in a closed position with a handle in an unactuated position;

FIG. 14 is a cross-sectional view of loading tool of FIG. 13, taken along section line 14-14 of FIG. 13;

FIG. 15 is a cross-sectional view of loading tool of FIG. 13, taken along section line 15-15 of FIG. 13;

FIG. 16 is a cross-sectional view of loading tool of FIG. 13, taken along section line 16-16 of FIG. 13;

FIG. 17 is a cross-sectional view of loading tool of FIG. 13, taken along section line 17-17 of FIG. 13;

FIG. 18 is a perspective view of the loading tool of FIG. 13, shown with the anvil of FIG. 2 positioned within the loading tool;

FIG. 19 is a cross-sectional view of the loading tool and the anvil of FIG. 18, taken along section line 19-19 of FIG. 18;

FIG. 20 is a cross-sectional view of the loading tool and the anvil of FIG. 18, taken along section line 20-20 of FIG. 18;

FIG. 21 is a cross-sectional view of the loading tool and the anvil of FIG. 19, taken along section line 21-21 of FIG. 19;

FIG. 22 is a cross-sectional view of the loading tool and the anvil of FIG. 21, during movement of the handle from the unactuated position to an actuated position;

FIG. 23 is a cross-sectional view of the loading tool and the anvil of FIG. 22, taken along section line 23-23 of FIG. 22;

FIG. 24 is a cross-sectional view of the loading tool and the anvil of FIG. 22, taken along section line 24-24 of FIG. 22;

FIG. 25 is a close-up view of the area of detail 25 indicated in FIG. 24;

FIG. 26 is a close-up view of the area of detail 26 indicated in FIG. 23;

FIG. 27 is a cross-sectional view of the loading tool and the anvil of FIG. 21, shown with the handle in the actuated position;

FIG. 28 is a cross-sectional view of the loading unit and anvil of FIG. 27, showing a proximal end portion of a surgical buttress engaging the anvil;

FIG. 29 is a cross-sectional view of the loading unit and anvil of FIG. 27, showing a distal end portion of the surgical buttress engaging the anvil;

FIG. 30 is a bottom view of the surgical buttress of FIGS. 28 and 29 loaded onto the anvil;

FIG. 31 is a close-up view of the area of detail 31 indicated in FIG. 30; and

FIG. 32 is a close-up view of the area of detail 32 indicated in FIG. 30.

### DETAILED DESCRIPTION

Aspects of the disclosure will now be described in detail with reference to the drawing figures wherein like reference numerals identify similar or identical elements. Throughout this description, the term “proximal” refers to a portion of a structure, or component thereof, that is closer to a user, and the term “distal” refers to a portion of the structure, or component thereof, that is farther from the user. Directional reference terms, such as “downwardly,” “upwardly,” and the like, are used to ease description of aspects of this disclosure and are not intended to have any limiting effect on the ultimate orientation of a structure or any part thereof.

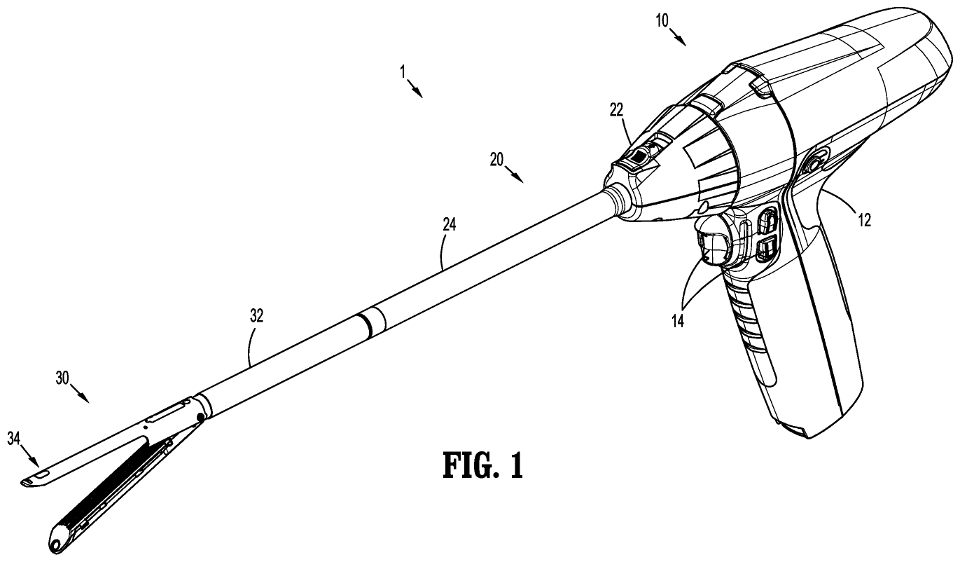

Referring now to FIG. 1, an exemplary surgical device or surgical stapling apparatus 1 is shown for use in stapling tissue in accordance with aspects of the disclosure. The surgical stapling apparatus 1 generally includes a handle assembly 10, an adapter assembly 20 extending distally from the handle assembly 10, and a loading unit 30 extending distally from the adapter assembly 20.

The surgical stapling apparatus 1 will further be described to the extent necessary to disclose aspects of the disclosure. For a detailed description of the structure and function of exemplary surgical devices, reference may be made to U.S. Pat. No. 10,426,468, the entire contents of which are incorporated herein by reference.

The handle assembly 10 includes a handle housing 12 housing a power-pack (not shown) configured to power and control various operations of the surgical stapling apparatus 1, and a plurality of actuators 14 (e.g., finger-actuated control buttons, knobs, toggles, slides, interfaces, and the like) for activating various functions of the surgical stapling apparatus 1. The adapter assembly 20 includes a knob housing 22 configured for operable connection to the handle assembly 10 and an elongate tubular body 24 configured for operable connection to the loading unit 30. Alternatively, the elongate tubular body 24 may be supported directly on the handle assembly 10 (e.g., permanently affixed to or integrally formed with the handle assembly).

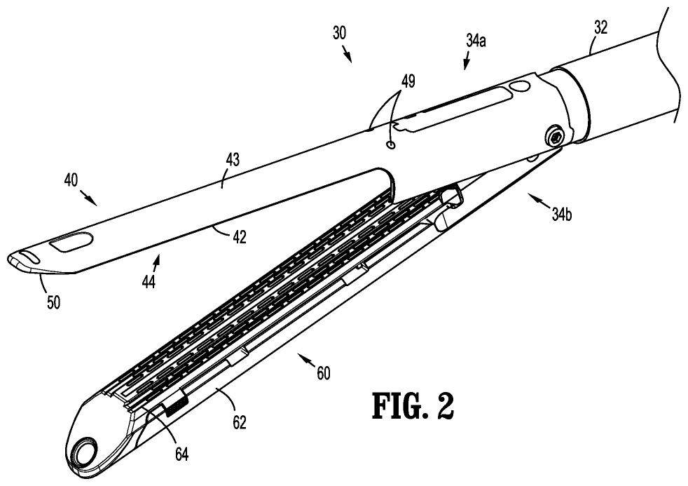

The loading unit 30 is a disposable loading unit (“DLU”) that is releasably secured to the elongated tubular body 24 and thus, replaceable with a new loading unit 30. The loading unit 30 may be a single use loading unit (“SULU”) that is used one time and then replaced to facilitate multiples uses of the surgical stapling apparatus 1 on a patient. For example, during a surgical procedure, the surgical stapling apparatus 1 can be used to staple and cut tissue, and the entire SULU is replaced after each staple and cut operation of the surgical stapling apparatus 1. The loading unit 30 may be a multi-use loading unit (“MULU”) that is re-useable a predetermined number of times. For example, during a surgical procedure, the surgical stapling apparatus 1 can be used to staple and cut tissue, and a reload assembly (e.g., a staple cartridge 64 as seen in FIG. 2) of the MULU is replaced after each staple and cut operation of the surgical stapling apparatus 1 a predetermined number of times before the entire MULU needs to be replaced. Alternatively, the loading unit 30 may be permanently affixed to the elongated tubular body 24.

As shown in FIGS. 1 and 2, the loading unit 30 includes a housing portion 32 and a tool or jaw assembly 34 including first and second jaw members 34a, 34b. The first jaw member 34a and/or the second jaw members 34b is pivotable with respect to the housing portion 32 such that the tool assembly 34 is movable between an open position in which the first and second jaw members 34a, 34b are spaced apart with respect to each other, and a closed position in which the first and second jaw members 34a, 34b are substantially adjacent each other. The first jaw member 34a of the tool assembly 34 includes an anvil assembly 40 (also referred to herein generally as an anvil) and the second jaw member 34b of the tool assembly 34 includes a staple cartridge assembly 60.

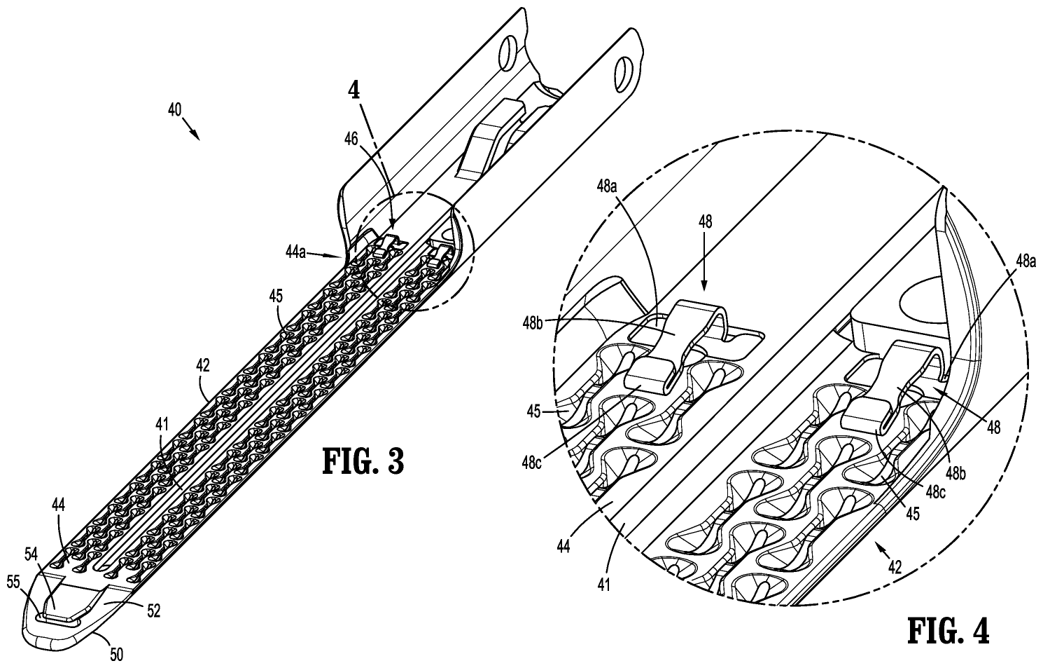

As shown in FIGS. 2 and 3, the anvil assembly 40 includes an anvil body 42 and an anvil tip 50 extending distally from the anvil body 42. The anvil body 42 has an outer surface 43 and an inner or tissue facing surface 44. The tissue facing surface 44 includes a central longitudinal slot 41 and staple forming pockets or cavities 45 defined therein. The central longitudinal slot 41 is configured for passage of a knife blade (not shown) therethrough during actuation of the surgical stapling apparatus 1 (FIG. 1). The staple forming pockets 45 are disposed in rows on opposed sides of the central longitudinal slot 41 and are configured to form staples (not shown) which are fired out of the staple cartridge assembly 60 during actuation of the surgical stapling apparatus 1 (FIG. 1). The anvil body 42 also includes a spring assembly 46 disposed at a proximal portion 44a of the tissue facing surface 44.

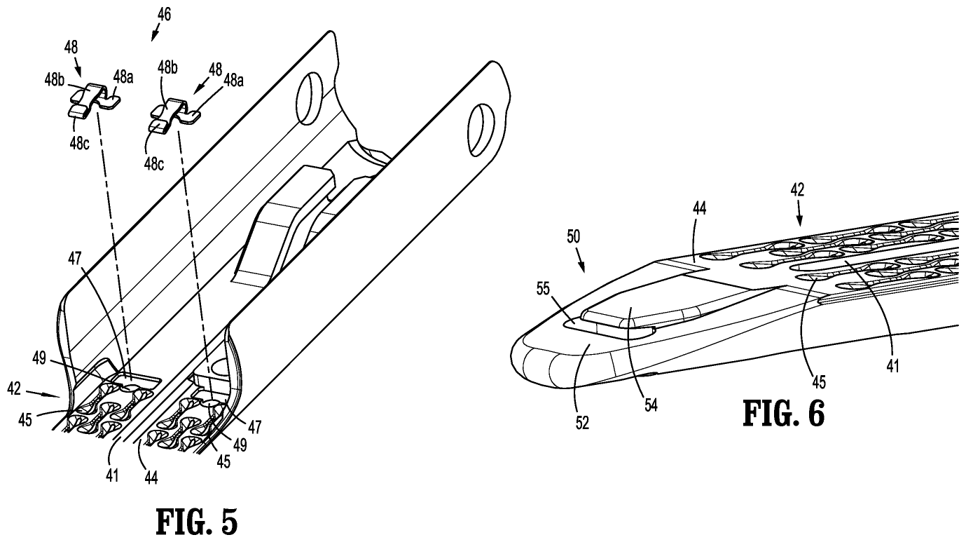

As shown in FIGS. 4 and 5, the spring assembly 46 includes a pair of springs 48, with each spring 48 disposed on opposed sides of the central longitudinal slot 41. Each spring 48 includes a base 48a, an arm 48b extending distally from the base 48a, and a finger 48c disposed at a distal end of the arm 48b. The base 48a is anchored or secured (e.g., welded or otherwise attached) to the tissue facing surface 44 of the anvil body 42 proximal to the staple forming pocket 45 and, in aspects, the base 48a is retained within a recess 47 defined in the tissue facing surface 44. The arm 48b extends distally from the base 48a and overlies a through-hole 49 (FIG. 5) that extends through the outer and tissue facing surfaces 43, 44 of the anvil body 42. The finger 48c is biased against (e.g., in direct contact with) the tissue facing surface 44 of the anvil body 42 adjacent the staple forming pocket 45. The arm 48b is flexible such that the finger 48c is movable away from (e.g., capable of being raised off of) the tissue facing surface 44 of the anvil body 42 upon application of a force thereto.

As shown in FIG. 6, in conjunction with FIG. 3, the anvil tip 50 extends from the anvil body 42 distal to the central longitudinal slot 41 and the staple forming pockets 45. The anvil tip 50 includes an inner surface 52 contiguous with and extending distally from the tissue facing surface 44 of the anvil body 42. The inner surface 52 may be angled or taper distally from the anvil body 42. The inner surface 52 includes a hook 54 extending distally over the inner surface 52 and, in aspects, the hook 54 extends over an opening 55 defined through the anvil tip 50.

With reference again to FIG. 2, the staple cartridge assembly 60 includes a cartridge carrier 62 configured and dimensioned to selectively receive and support a staple cartridge 64 therein. A drive assembly (not shown) is supported in the anvil and staple cartridge assemblies 40, 60 and slidable relative thereto to fire staples (not shown) from the staple cartridge 64 and to cut tissue disposed between the anvil and staple cartridge assemblies 40, 60 during actuation of the surgical stapling apparatus 1 (FIG. 1).

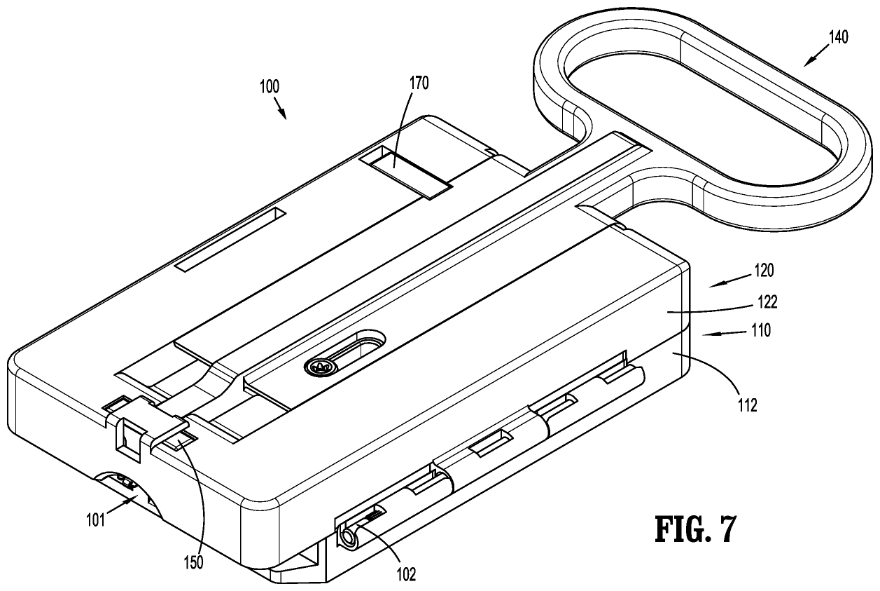

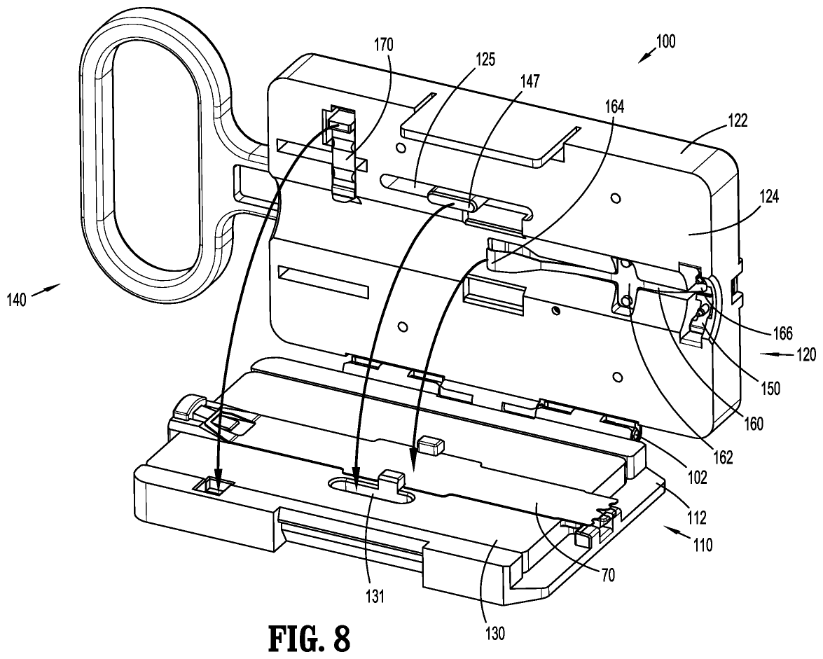

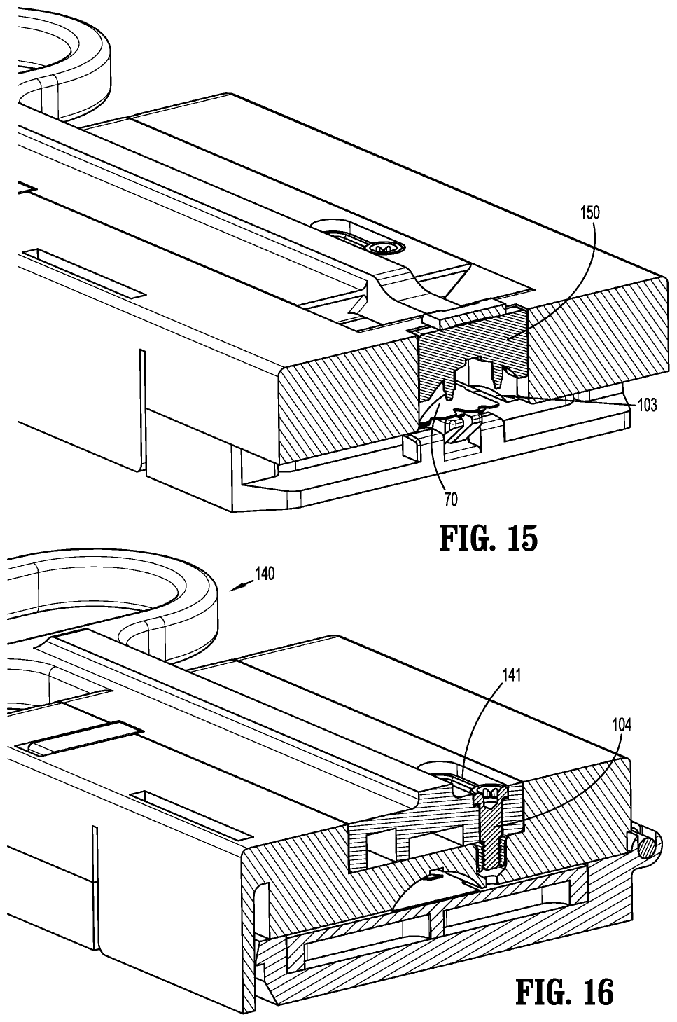

Turning now to FIGS. 7 and 8, a surgical buttress loading tool 100 (also referred to herein generally as a loading tool) is shown for loading a surgical buttress 70 (FIG. 8) onto the anvil 40 (FIG. 2). The loading tool 100 includes a first housing portion 110 hingedly connected to a second housing portion 120 (e.g., about a hinge pin 102) such that the loading tool 100 is movable between a closed position (FIG. 7) and an open position (FIG. 8). In the closed position, the loading tool 100 defines a proximal opening 101 into a cavity 103 (FIG. 15) defined between the first and second housing portions 110, 120 that is sized and shaped to receive the anvil 40 (FIG. 2). In the open position, a buttress cartridge 130 may be placed into or removed from the loading tool 100.

As shown in FIGS. 7-9, the first housing portion 110 includes a first housing body 112 supporting the buttress cartridge 130 and the surgical buttress 70 thereon, and the second housing portion 120 includes a second housing body 122 supporting a handle 140, an actuator 150, an applicator spring 160, and an interlock 170. The surgical buttress 70 is releasably retained on the buttress cartridge 130 which is removably positionable within the first housing portion 110 and slidable relative to the first housing body 112. The handle 140 is slidable relative to the second housing body 122 between unactuated and actuated positions, and is configured to move the actuator 150 into engagement with the spring assembly 46 (FIG. 3) of the anvil 40 when the anvil 40 is positioned within the cavity 103 (FIG. 15) of the loading tool 100 and to slide the buttress cartridge 130 towards the anvil 40 for loading the surgical buttress 70 thereon. The applicator spring 160 is configured to hold the surgical buttress 70 against the buttress cartridge 130 until the anvil 40 (FIG. 2) is positioned into the loading tool 100 and to bias the actuator 150 in a retracted position within the second housing body 122 until the handle 140 moves the actuator 150 to an extended position. The interlock 170 is configured to lock the handle 140 and the buttress cartridge 130 from movement until the anvil 40 (FIG. 2) is positioned within the loading tool 100.

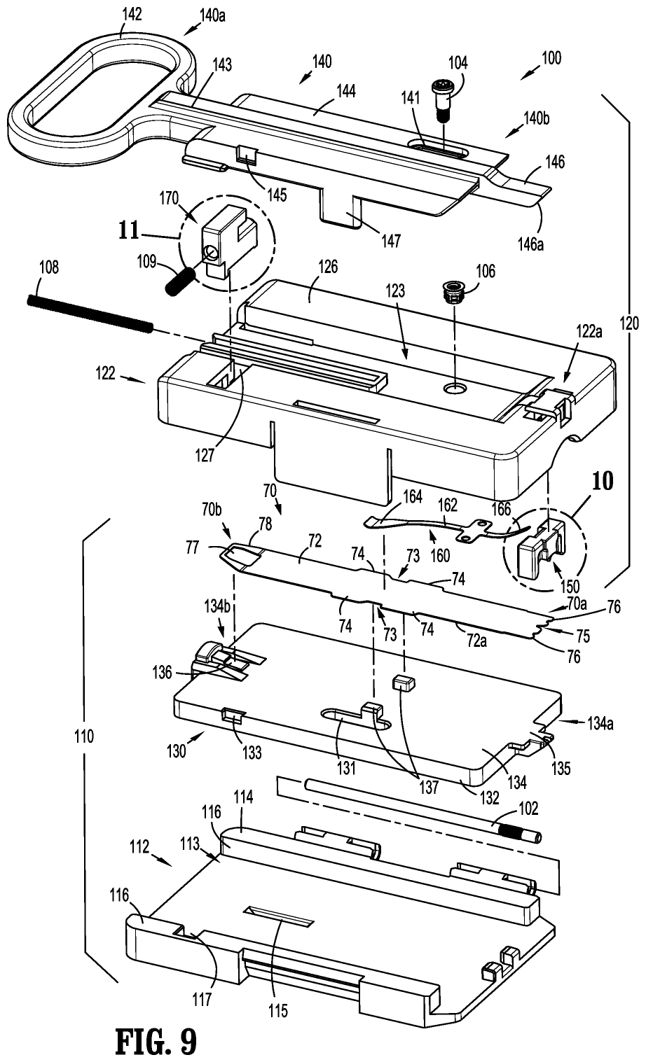

As seen in FIG. 9, the surgical buttress 70 includes a buttress body 72 having a pair of tabs 74 extending laterally from each longitudinal side 72a of the buttress body 72 in spaced relation relative to each other to define a recess 73 on opposed sides of the buttress body 72. The recesses 73 are configured to engage protrusions 137 of the buttress cartridge 130 when the surgical buttress 70 is positioned on the buttress cartridge 130. A proximal end portion 70a of the surgical buttress 70 includes a v-shaped notch 75 defined therein and proximal tabs 76 disposed on opposed sides of the v-shaped notch 75. A distal end portion 70b of the surgical buttress 70 includes a window 77 defined in a distal tab 78.

The buttress cartridge 130 includes a cartridge body 132 having an inner surface 134 that faces an inner surface 124 (FIG. 8) of the second housing portion 120. The inner surface 134 is configured to support and releasably retain the surgical buttress 70 thereon. The inner surface 134 includes a proximal end portion 134a having a central bump-out 135 upon which the proximal end portion 70a of the surgical buttress 70 is placed, a distal end portion 134b including a ramped or stepped surface 136 over which the distal end portion 70b of the surgical buttress 70 is placed, and a pair of opposed and spaced protrusions 137 extending upwardly towards the second housing portion 120 between which the buttress body 72 of the surgical buttress 70 is placed. The central bump-out 135 is configured so that the proximal tabs 76 of the surgical buttress 70 do not overlie the inner surface 134 of the buttress cartridge 130, the stepped surface 136 is configured so that the distal tab 78 of the surgical buttress 70 is disposed at an angle with respect to the buttress body 72, and the protrusions 137 are shaped for positioning within the recesses 73 of the surgical buttress 70 to aid in proper alignment of the surgical buttress 70 on the buttress cartridge 130 and to minimize movement of the surgical buttress 70 relative to the buttress cartridge 130.

The buttress cartridge 130 further includes an elongated slot 131 defined through the inner surface 134 which is configured to receive a tab 147 of the handle 150 when the loading tool 100 is in the closed position, and a notch 133 defined in an outer edge thereof that is configured to engaged the interlock 170 when the loading tool 100 is in the closed position and the anvil 40 (FIG. 2) is not disposed within the loading tool 100.

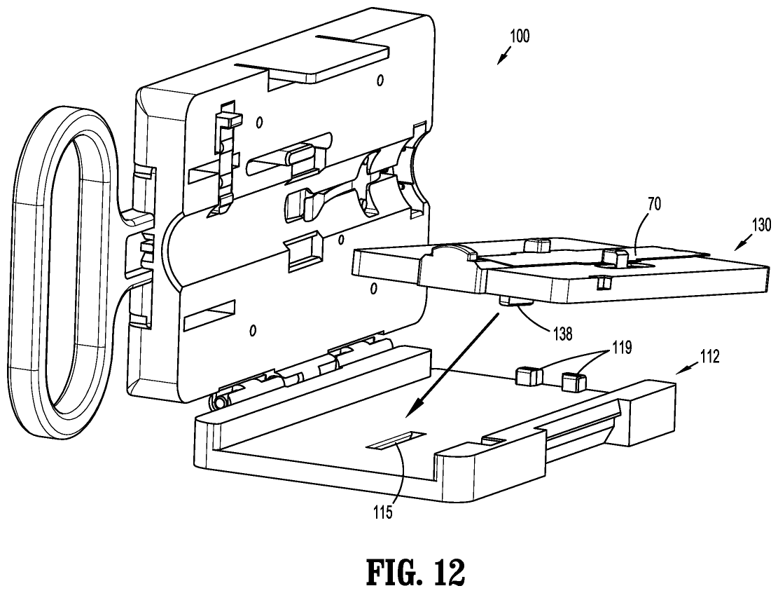

The buttress cartridge 130 is removably positionable within a channel 113 of the first housing body 112 that is defined in an inner surface 114 between a pair of opposed rails 116. The buttress cartridge 130 includes a tab 138 (FIG. 12) configured for slidable receipt within an elongated slot 115 of the first housing body 112. The first housing body 112 further includes a notch 117 defined in one of the rails 116 that is aligned with the notch 133 of the buttress cartridge 130 and into which the interlock 170 is moved when the anvil 40 (FIG. 2) is positioned within the loading tool 100.

With continued reference to FIG. 9, the handle 140 includes a head 142 at a distal end 140a thereof, a neck 143 extending proximally from the head 142, a handle body 144 extending proximally from the neck 143, and a tail 146 at a proximal end 140b thereof. The head 142 extends outwardly beyond the second housing body 122 and is configured for grasping by a user. The handle body 144 is secured within a channel 123 defined in an outer surface 126 of the second housing body 122 and is longitudinally slidable relative thereto. The handle body 144 includes a longitudinally extending slot 141 defined therethrough in which a screw 104 is positioned. The screw 104 is engaged (e.g., threadingly engaged) with an insert 106 disposed within the second housing body 122 to limit longitudinal movement of the handle 140 relative to the second housing body 122. The handle body 144 further includes a notch 145 defined in a longitudinal side thereof that is configured to engage the interlock 170 when the anvil 40 (FIG. 2) is not disposed within the loading tool 100, and a tab 147 extending downwardly form the handle body 144 and through an elongate slot 125 (FIG. 8) defined through the second housing body 122. When the loading tool 100 is in the closed position, the tab 147 of the handle body 144 extends into the elongated slot 131 of the buttress cartridge 130. The tail 146 has a sloped proximal end 146a that is positioned adjacent to (e.g., against) the actuator 150 and is configured to cam the actuator 150 downwardly towards the first housing portion 110 upon movement of the handle 140 to the actuated position. The handle 140 is biased in the unactuated position (FIG. 7) by a spring 108 positioned within the second housing body 122.

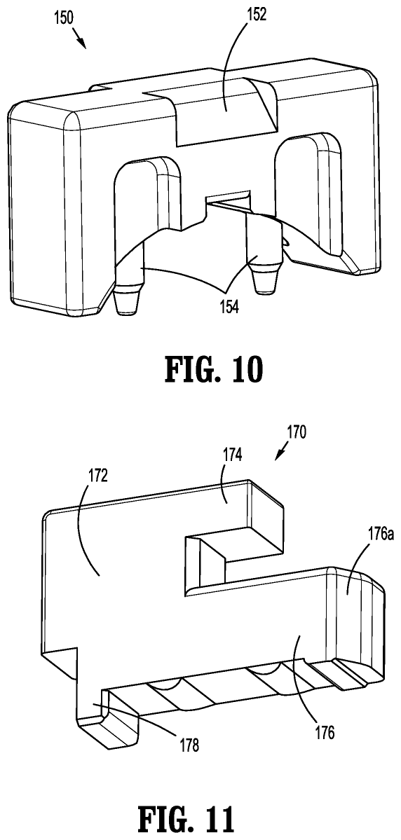

The actuator 150 is disposed within a proximal end portion 122a of the second housing body 122 and is movable into the cavity 103 (FIG. 15) defined between the first and second housing bodies 112, 122. As shown in FIG. 10, the actuator 150 includes a ramped surface 152 configured to engage the sloped proximal end 146a (FIG. 9) of the tail 146 of the handle 140 such that when the handle 140 is moved proximally, the tail 146 engages the actuator 150 and pushes the actuator 150 downwardly towards the first housing portion 110. The actuator 150 further includes a pair of pins 154 sized and shaped to extend through the through-holes 49 (FIG. 2) of the anvil 40 when the anvil 40 is inserted into the loading tool 100 and to contact and move the springs 48 (FIG. 4) of the spring assembly 46.

With continued reference to FIG. 9, the applicator spring 160 includes a spring body 162 secured to the inner surface 124 (FIG. 8) of the second housing portion 122. The applicator spring 160 includes a spring arm 164 extending distally from the spring body 162 that is configured to engage the surgical buttress 70 and hold the surgical buttress 70 down against the buttress cartridge 130 until the anvil 40 (FIG. 2) is positioned within the loading tool 100, and a spring leg 166 extending proximally from the spring body 162 that is configured to engage the actuator 150 between the pins 154 and bias the actuator 150 into the retracted state (FIG. 8).

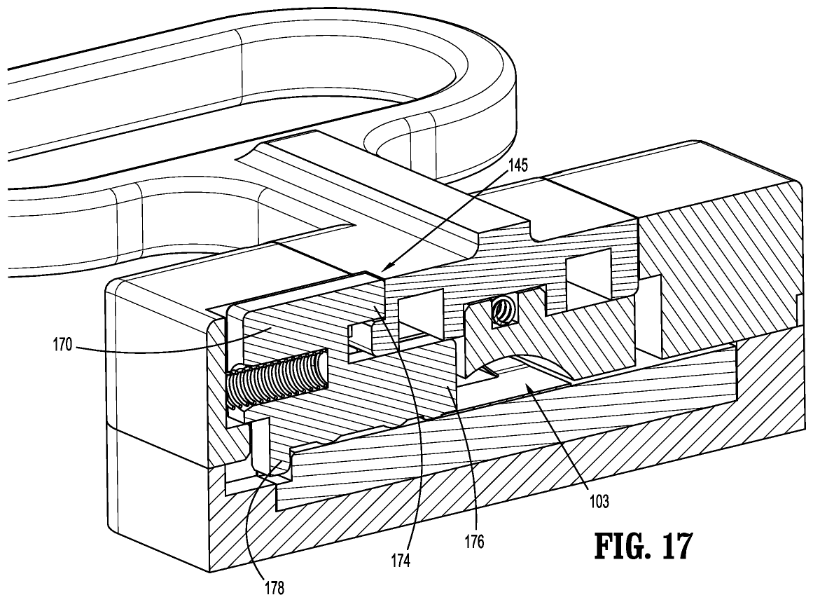

The interlock 170 is disposed within a laterally extending slot 127 defined in the second housing body 122 adjacent to the handle 140. As shown in FIG. 11, in conjunction with FIG. 9, the interlock 170 includes an interlock body 172 including first and second arms 174, 176 extending laterally therefrom in spaced relation relative to each other and a leg 178 extending downwardly towards the first housing portion 110. The interlock 170 is biased into a locked position by a spring 109. In the locked position, the first arm 174 is disposed within the notch 145 of the handle 140, the second arm 176 extends into the cavity 103 (FIG. 17) defined between the first and second housing portions 110, 120, and the leg 178 is disposed within the notch 133 of the buttress cartridge 130 so that the handle 140 cannot be moved. When an anvil 40 (FIG. 2) is inserted into the cavity 103 (FIG. 17), the anvil 40 cams against a beveled surface 176a (FIG. 11) of the second arm 176 of the interlock 170, pushing the interlock 170 laterally outwardly to an unlocked position so that the first arm 174 moves out of the notch 145 of the handle and the leg 178 moves out of the notch 133 of the buttress cartridge 130 and into the notch 117 of the first housing body 112, thereby releasing the handle 140.

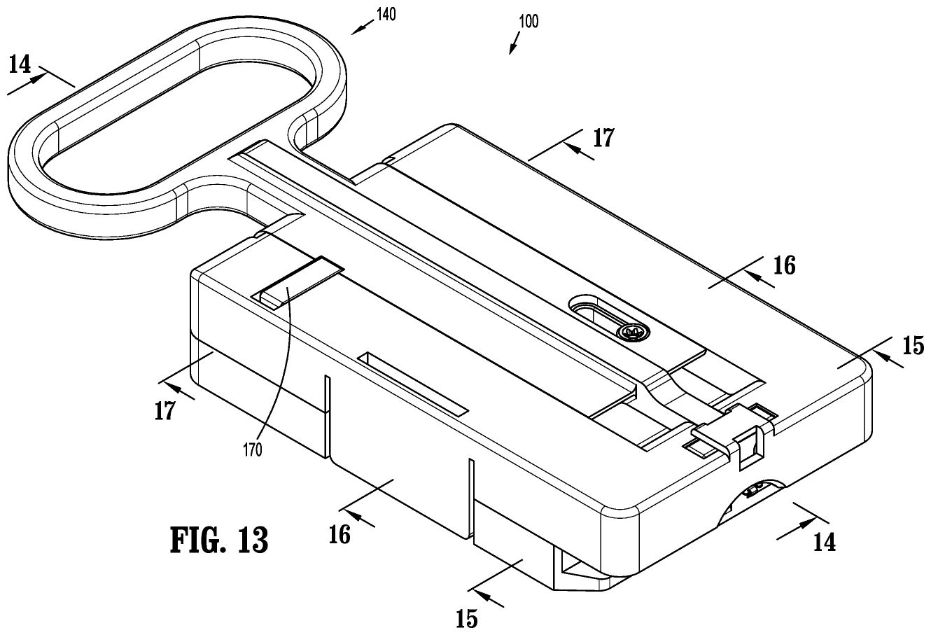

Turning now to FIG. 12, the loading tool 100 is loaded with a surgical buttress 70 by positioning a buttress cartridge 130 containing the surgical buttress 70 (e.g., the buttress cartridge may be pre-loaded with the surgical buttress by, for example, the manufacturer) within the loading tool 100. The buttress cartridge 130 is loaded by placing the tab 138 of the buttress cartridge 130 into the elongated slot 115 of the first housing portion 112 and positioning a proximal end of the buttress cartridge 130 against protrusions 119 disposed at a proximal end of the first housing body 112. It should be understood that a spent buttress cartridge 130 may be removed from the loading tool 100 and replaced with a fresh buttress cartridge 130 for re-use of the loading tool 100. Once loaded with a buttress cartridge 130 containing a surgical buttress 70, the loading tool 100 is closed for use, as shown in FIG. 13.

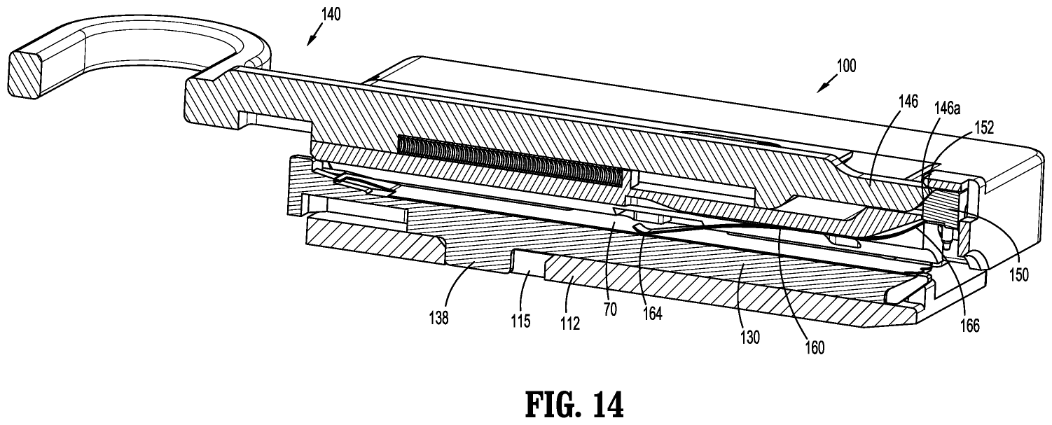

In the closed position, as shown in FIGS. 13 and 14, the tab 138 of the buttress cartridge 130 is disposed within the elongated slot 115 of the first housing body 112, as described above, and the spring arm 164 of the applicator spring 160 contacts and holds the surgical buttress 70 down against the buttress cartridge 130. Further, the spring leg 166 of the applicator spring 160 biases the actuator 150 in the retracted position, as seen in FIGS. 14 and 15, so that the actuator 150 is positioned above the cavity 103 and the surgical buttress 70 is disposed distal to the actuator 150. With continued reference to FIGS. 13 and 14, the handle 140 is disposed in the unactuated position with the sloped proximal end 146a of the tail 146 positioned adjacent to the ramped surface 152 of the actuator 150 and, as also seen in FIG. 16, the screw 104 disposed in a proximal-most portion of the longitudinally extending slot 141. As shown in FIGS. 13 and 17, the interlock 170 is in the locked state with the first arm 174 of the interlock 170 disposed within the notch 145 of the handle 140 so that the handle 140 cannot be moved distally without an anvil 40 (FIG. 2) inserted into cavity 103 as the second arm 176 of the interlock 170 extends partially into the cavity 103.

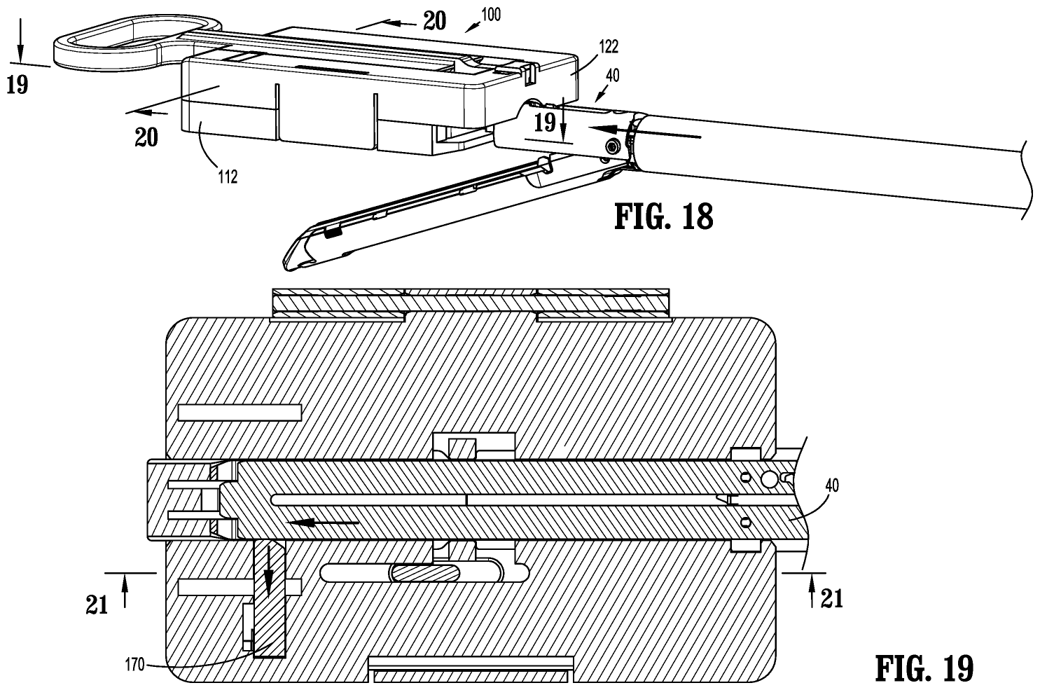

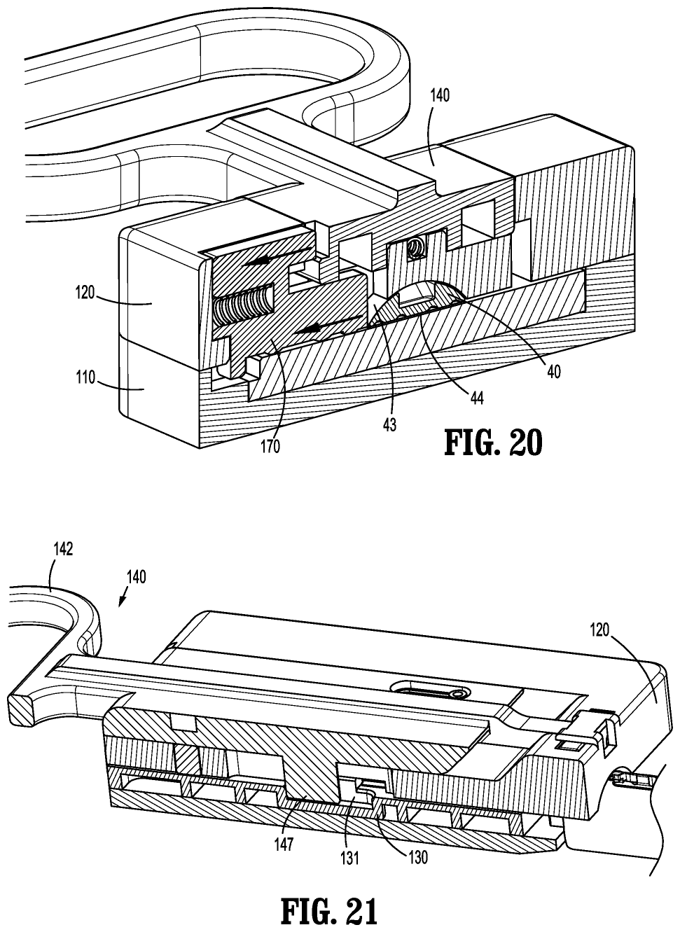

The loading tool 100 is configured to engage the anvil 40 for loading the surgical buttress 70 onto the anvil 40 (e.g., transferring the surgical buttress 70 from the loading tool 100 to the anvil 40). The anvil 40 is aligned with the proximal opening 101 of the loading tool 100 and then inserted through the proximal opening 101 and into the cavity 103 defined between the first and second housing bodies 112, 122 until the anvil 40 is fully inserted into the loading tool 100, as shown in FIG. 18. The proximal opening 101 and corresponding cavity 103 of the loading tool 100 are shaped to correspond with the shape of the anvil 40 and thus, is configured to slidably receive the anvil 40 in an orientation in which the tissue facing surface 44 of the anvil 40 faces the first housing portion 110 and the outer surface 43 faces the second housing portion 120, as seen in FIG. 20.

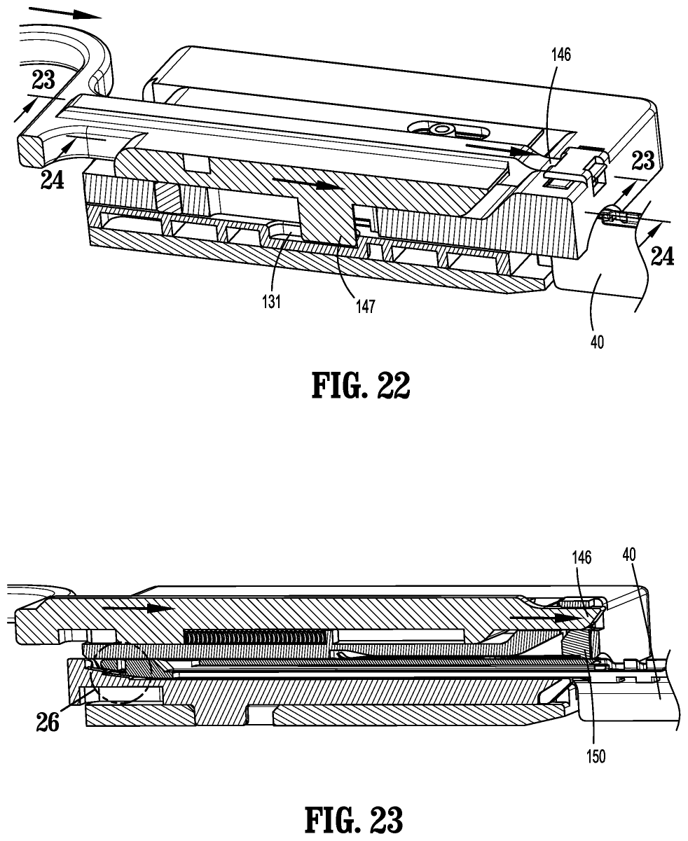

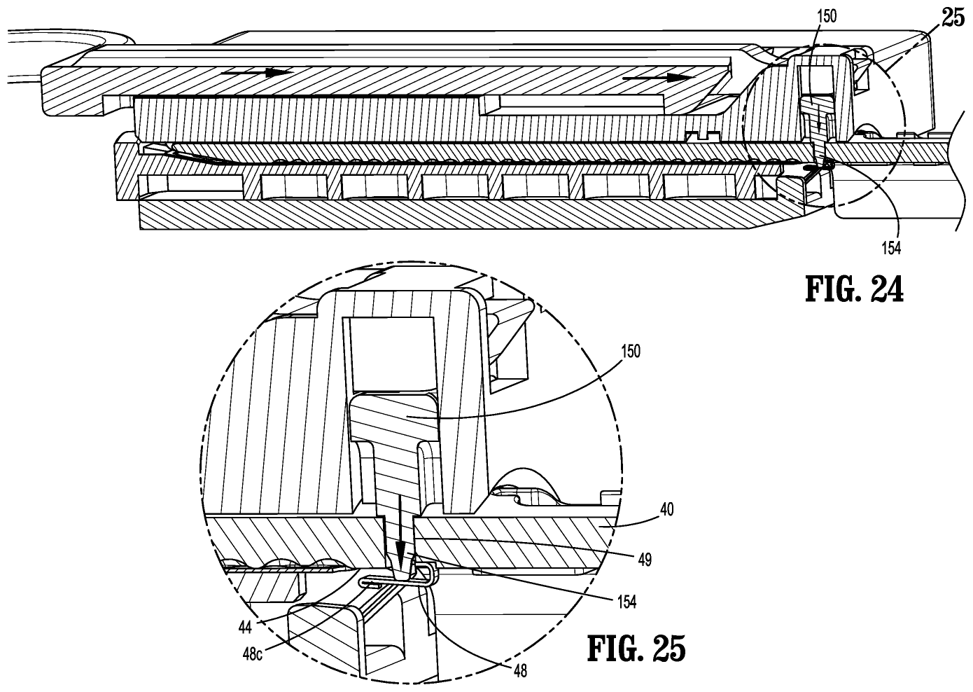

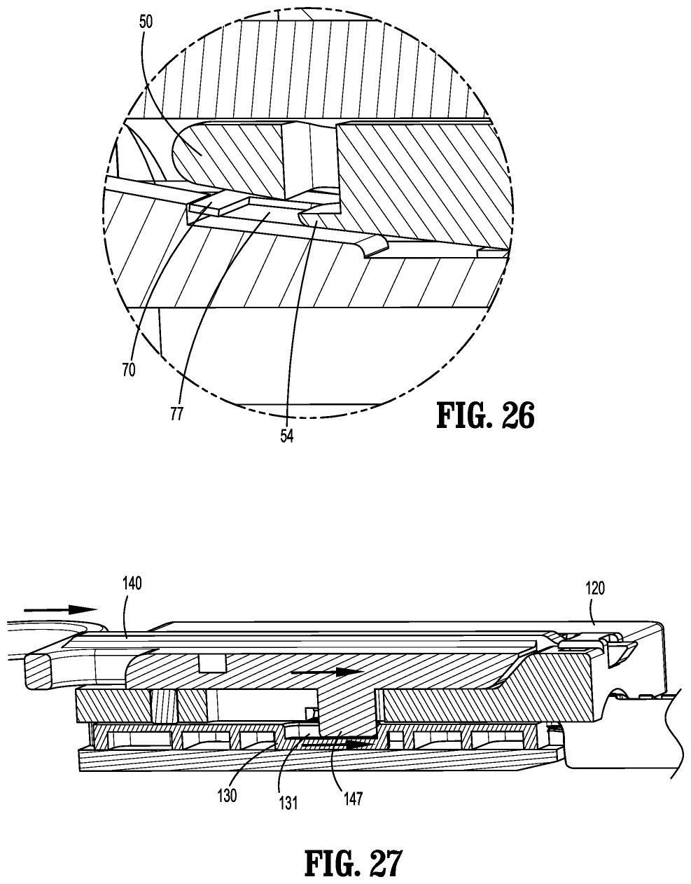

As shown in FIGS. 19 and 20, once the anvil 40 is fully inserted, the anvil 40 cams the interlock 170 laterally outwardly so that the handle 140 is free to move from the unactuated position to the actuated position. As shown in FIG. 21, in the unactuated position, the tab 140 of the handle 140 is initially disposed in a distal portion of the elongated slot 131 defined in the buttress cartridge 130. As the handle 140 is moved (e.g., by pushing the head 142 of the handle 140 towards or into the second housing portion 120), the tab 147 travels (e.g., slides) into a proximal portion of the elongated slot 131 before moving the buttress cartridge 130. During this initial sliding movement, with the tab 147 of the handle 140 traveling within the elongated slot 131 of the buttress cartridge 130, as seen in FIGS. 22 and 23, the tail 146 of the handle 140 cams the actuator 150 down towards the anvil 40. As shown in FIGS. 24 and 25, the pins 154 of the actuator 150, which are aligned with the through-holes 49 defined in the anvil 40, travel down through the through-holes 49 of the anvil 40 and push the springs 48 so that the fingers 48c of the springs 48 are spaced from the tissue facing surface 44 of the anvil 40. As further seen in FIG. 26, the hook 54 of the anvil tip 50 aligns with the window 77 of the surgical buttress 70.

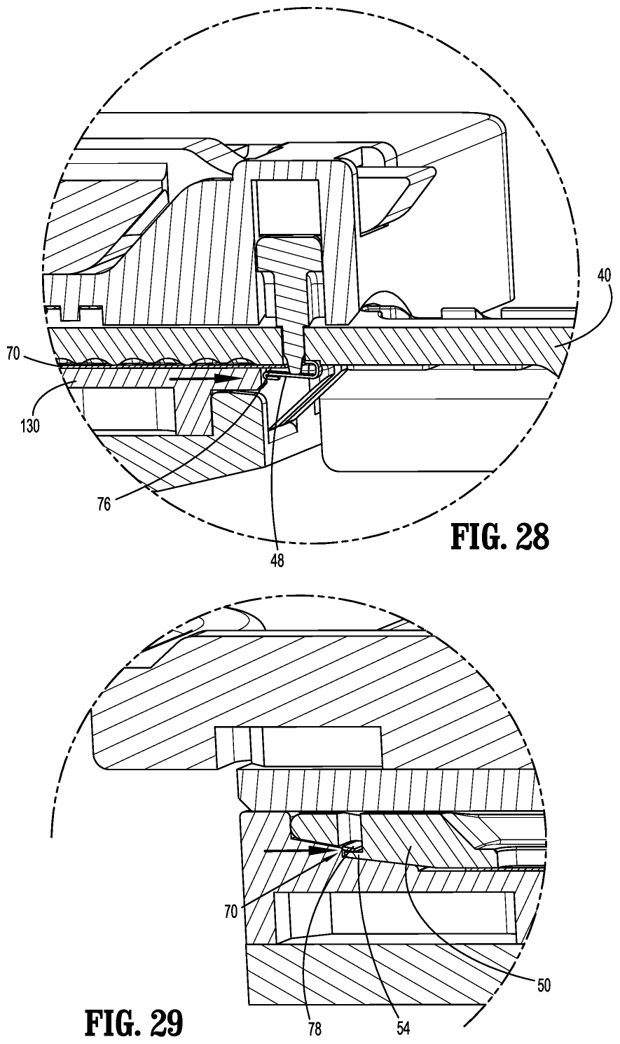

Upon further movement of the handle 140 proximally into the second housing portion 120 to the actuated position, as shown in FIG. 27 (after the tab 147 of the handle 140 engages a proximal end of the elongated slot 131 of the buttress cartridge 130), the handle 140 moves the buttress cartridge 130 proximally. As the buttress cartridge 130 moves proximally, the surgical buttress 70 is also moved proximally so that the proximal tabs 76 of the surgical buttress 70 are moved under the springs 48 of the anvil 40, as shown in FIG. 28, and the distal tab 78 of the surgical buttress 70 is moved into engagement with the hook 54 of the anvil tip 50, as shown in FIG. 29.

The handle 140 may then be moved back to the unactuated position (e.g., the handle 140 may be pulled back or released so that the spring 108 drive movement distally). During this distal movement, the tail 146 of the handle 140 disengages the actuator 150 thereby freeing the actuator 150 to move back to its retracted position and out of engagement with the anvil 40, releasing the springs 48 so that the proximal tabs 76 are secured to the anvil 40. The distal tab 78 is also secured to the anvil 40 by the hook 54. Further, during this distal movement, the buttress cartridge 130 is retracted distally and away from the surgical buttress 70 which is now secured to the anvil 40. Once the handle is back in the unactuated position, the anvil 40 is removed from the loading tool 100 and the interlock 170 re-engages the handle 140 and the buttress cartridge 130.

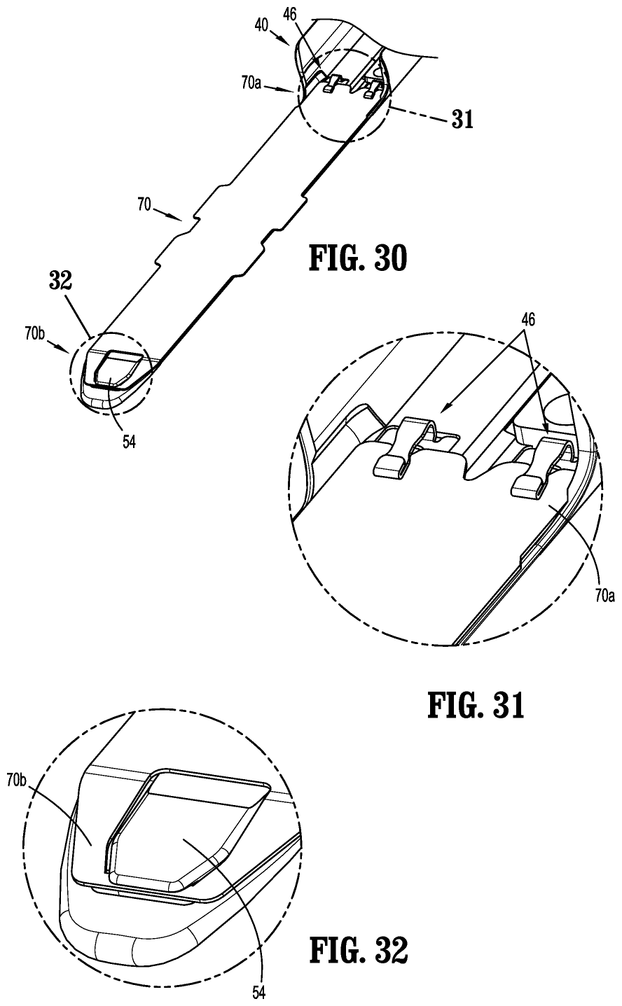

As shown in FIGS. 30-32, the anvil assembly 40 is loaded with the surgical buttress 70. The proximal end portion 70a of the surgical buttress 70 is retained on the anvil 40 via the spring assembly 46 and the distal end portion 70b of the surgical buttress 70 is retained on the anvil 40 via the hook 54. The surgical stapling apparatus 1 (FIG. 1), with the anvil assembly 40 loaded with the surgical buttress 70, is ready for use. In aspects, the staple cartridge assembly 60 is pre-loaded and/or loaded with a surgical buttress by any suitable attachment feature within the purview of those skilled in the art, such as, for example, mechanical attachment features (e.g., a suture), chemical attachment features (e.g., adhesive), and/or attachment methods (e.g., welding).

In operation, with the loading unit 30 loaded with the surgical buttress 70, as described above, the surgical stapling apparatus 1 is used in accordance with methods known by those skilled in the art. Once the anvil and staple cartridge assemblies 40, 60 are clamped onto tissue, the surgical stapling apparatus 1 is fired, thereby stapling the surgical buttress 70 to the tissue. During firing, a knife (not shown) travels distally through the tool assembly 34 (through the central longitudinal slot 41 of the anvil 40) and substantially simultaneously cuts and divides the tissue and the surgical buttress 70 disposed between the rows of formed staples. When firing is complete and the anvil and staple cartridge assemblies 40, 60 are unclamped, the surgical buttress 70, which is now stapled to the tissue, pulls away from the anvil 40, and the tool assembly 34 can be removed from the surgical site. The used staple cartridge 64 may then be removed from the tool assembly 34 and replaced with a new staple cartridge 64. A new surgical buttress 70 may be installed onto the anvil 40, as needed or desired, as described above.

While illustrated as being used on a handheld powered surgical device hereinabove, it is contemplated, and within the scope of the disclosure for the surgical buttress loading tools to be configured for use with handheld manually-actuated surgical devices, as well as other electrosurgical instruments. For example, the surgical buttress loading tools may be used on handheld manually actuated surgical devices, such as those shown and described in U.S. Pat. Nos. 4,473,077, 5,915,616, 5,964,394, 6,330,965, 7,128,253, and 7,334,717, the entire contents of each of which are incorporated herein by reference. As another example, the surgical buttress loading tools may be used on robotic surgical systems, such as the robotic surgical system shown and described in U.S. Pat. No. 8,828,023, the entire contents of which are incorporated herein by reference.

While aspects of the disclosure have been shown in the drawings, it is not intended that the disclosure be limited thereto, as it is intended that the disclosure be as broad in scope as the art will allow and that the specification be read likewise. It is to be understood, therefore, that the disclosure is not limited to the precise aspects described, and that various other changes and modifications may be effected by one skilled in the art without departing from the scope or spirit of the disclosure. Therefore, the above description should not be construed as limiting, but merely as exemplifications of aspects of the disclosure. Thus, the scope of the disclosure should be determined by the appended claims and their legal equivalents, rather than by the examples given.

1-20. (canceled)

21. A surgical loading unit for a surgical device, the surgical loading unit comprising:
an anvil including an anvil body defining a longitudinally extending knife slot and at least one longitudinally extending row of staple forming pockets disposed on each side of the knife slot, wherein each row of staple forming pockets includes a distal-most staple forming pocket and a proximal-most staple forming pocket; and
a spring assembly disposed on each side of the knife slot, wherein each spring assembly is axially aligned with a respective row of staple forming pockets, and wherein at least a portion of each spring assembly extends distally of the proximal-most staple forming pocket.

22. The surgical loading unit according to claim 21, wherein the at least one longitudinally extending row of staple forming pockets includes:
at least two rows of longitudinally extending rows of staple forming pockets disposed on a first side of the knife slot, wherein:
a first row of the at least two rows of staple forming pockets disposed on the first side of the knife slot defines the proximal-most staple forming pocket;
a second row of the at least two rows of staple forming pockets disposed on the first side of the knife slot is off-set distally of the first row of the at least two rows of staple forming pockets;
at least a portion of each spring assembly extends distally of the proximal-most staple forming pocket of the first row of the at least two rows of staple forming pockets disposed on the first side of the knife slot; and
the at least a portion of each spring assembly is axially aligned proximally of the second row of the at least two rows of staple forming pockets disposed on the first side of the knife slot; and
at least two rows of longitudinally extending rows of staple forming pockets disposed on a second side of the knife slot, wherein:
a first row of the at least two rows of staple forming pockets disposed on the second side of the knife slot defines the proximal-most staple forming pocket;
a second row of the at least two rows of staple forming pockets disposed on the second side of the knife slot is off-set distally of the first row of the at least two rows of staple forming pockets;
at least a portion of each spring assembly extends distally of the proximal-most staple forming pocket of the first row of the at least two rows of staple forming pockets disposed on the second side of the knife slot; and
the at least a portion of each spring assembly is axially aligned proximally of the second row of the at least two rows of staple forming pockets disposed on the second side of the knife slot.

23. The surgical loading unit according to claim 21, wherein each spring assembly includes a spring having an arm extending over a through-hole defined in the anvil body.

24. The surgical loading unit according to claim 21, wherein the anvil includes an anvil tip extending distally from the anvil body, the anvil tip including a hook.

25. The surgical loading unit according to claim 21, wherein the knife slot is centrally located in the anvil body.

26. A surgical device, comprising:
the surgical loading unit according to claim 21; and
a staple cartridge assembly operatively connected to the anvil, wherein the anvil and the staple cartridge assembly are pivotally connected to one another.

27. The surgical device according to claim 26, wherein the staple cartridge assembly includes a knife slot for operative association with the knife slot of the anvil.

28. The surgical device according to claim 27, wherein the staple cartridge assembly includes a row of staple retaining pockets corresponding to each row of staple forming pockets of the anvil, and wherein each staple retaining pocket includes a staple loaded therein.

29. The surgical device according to claim 26, further comprising a surgical buttress overlying the staple forming pockets of the anvil, wherein a proximal end of the surgical buttress is captured between the anvil body and the spring assembly.

30. A surgical loading unit for a surgical device, the surgical loading unit comprising:
an anvil including:
an anvil body defining a longitudinally extending knife slot and at least one longitudinally extending row of staple forming pockets disposed on each side of the knife slot, wherein each row of staple forming pockets includes a distal-most staple forming pocket and a proximal-most staple forming pocket; and
a spring assembly disposed on each side of the knife slot, wherein each spring assembly is axially aligned with a respective row of staple forming pockets, and wherein at least a portion of each spring assembly extends distally of the proximal-most staple forming pocket;
a staple cartridge assembly operatively connected to the anvil, wherein the anvil and the staple cartridge assembly are pivotally connected to one another; and
a surgical buttress overlying the staple forming pockets of the anvil, wherein a proximal end of the surgical buttress is captured between the anvil body and the spring assembly.

31. The surgical loading unit according to claim 30, wherein the at least one longitudinally extending row of staple forming pockets of the anvil includes:
at least two rows of longitudinally extending rows of staple forming pockets disposed on a first side of the knife slot, wherein:
a first row of the at least two rows of staple forming pockets disposed on the first side of the knife slot defines the proximal-most staple forming pocket;
a second row of the at least two rows of staple forming pockets disposed on the first side of the knife slot is off-set distally of the first row of the at least two rows of staple forming pockets;
at least a portion of each spring assembly extends distally of the proximal-most staple forming pocket of the first row of the at least two rows of staple forming pockets disposed on the first side of the knife slot; and
the at least a portion of each spring assembly is axially aligned proximally of the second row of the at least two rows of staple forming pockets disposed on the first side of the knife slot; and
at least two rows of longitudinally extending rows of staple forming pockets disposed on a second side of the knife slot, wherein:
a first row of the at least two rows of staple forming pockets disposed on the second side of the knife slot defines the proximal-most staple forming pocket;
a second row of the at least two rows of staple forming pockets disposed on the second side of the knife slot is off-set distally of the first row of the at least two rows of staple forming pockets;
at least a portion of each spring assembly extends distally of the proximal-most staple forming pocket of the first row of the at least two rows of staple forming pockets disposed on the second side of the knife slot; and
the at least a portion of each spring assembly is axially aligned proximally of the second row of the at least two rows of staple forming pockets disposed on the second side of the knife slot.

32. The surgical loading unit according to claim 30, wherein each spring assembly includes a spring having an arm extending over a through-hole defined in the anvil body.

33. The surgical loading unit according to claim 30, wherein the anvil includes an anvil tip extending distally from the anvil body, the anvil tip including a hook.

34. The surgical loading unit according to claim 30, wherein the knife slot is centrally located in the anvil body.

35. A surgical device, comprising:
the surgical loading unit according to claim 30; and
a staple cartridge assembly operatively connected to the anvil, wherein the anvil and the staple cartridge assembly are pivotally connected to one another.

36. The surgical device according to claim 35, wherein the staple cartridge assembly includes a knife slot for operative association with the knife slot of the anvil.

37. The surgical device according to claim 36, wherein the staple cartridge assembly includes a row of staple retaining pockets corresponding to each row of staple forming pockets of the anvil, and wherein each staple retaining pocket includes a staple loaded therein.
